(19) United States
(12) Patent Application Publication
(10) Pub. No.: US 2021/0145885 A1
(43) Pub. Date: May 20, 2021

(54) MATERIALS AND METHODS FOR TREATING VITILIGO

(71) Applicant: NORTHWESTERN UNIVERSITY, Evanston, IL (US)

(72) Inventors: I. Caroline Le Poole, Downers Grove, IL (US); Zhussipbek Mukhatayev, Evanston, IL (US); Richard Paul Junghans, Boston, MA (US)

(21) Appl. No.: 17/072,939

(22) Filed: Oct. 16, 2020

Related U.S. Application Data
(60) Provisional application No. 62/915,945, filed on Oct. 16, 2019.

(51) Int. Cl. A61K 35/17; A61K 9/00; A61K 38/17; A61P 37/06; A61P 17/00

(52) U.S. Cl. CPC A61K 35/17; A61K 9/0019; A61P 17/00; A61P 37/06; A61K 38/177

(57) ABSTRACT

The present disclosure provides materials and methods for the treatment of autoimmune diseases (including vitiligo).

### CROSS-REFERENCE TO RELATED APPLICATION

The present application claims the benefit of priority to U.S. Provisional Application No. 62/915,945, filed Oct. 16, 2019, the disclosure of which is incorporated herein by reference in its entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 2019-172_Seqlisting.txt; Size: 2,658 bytes, created: Oct. 16, 2020.

### BACKGROUND

Vitiligo is an autoimmune skin condition resulting from T cell mediated loss of melanocytes in the skin. Autoimmunity has been proposed as a cause for progressive depigmentation. As a result of the loss of melanocytes, white patches of skin appear on different parts of the body. Any part of the body may be affected. Hair pigmentation often remains unaffected, and ocular and auditory abnormalities are rare. In the United States, approximately 2.6 million people have the disorder, and about 1 percent of the world's population is affected by this disease. Vitiligo is more pronounced on darker skin. It affects people of both sexes, though approximately 25% more females are affected, and it affects all ethnicities. Vitiligo can begin at any age, though about fifty percent of people with vitiligo develop it before the age of twenty-five. Vitiligo can cause extreme distress to sufferers because of its unusual appearance.

Treatment options currently available include medical, surgical, and other interventions. However, individual treatments are not appropriate for all patients, and many treatments have unwanted side effects. Current treatments can require substantial time and effort to achieve significant depigmentation. Treatments are aimed at restoring color to the white patches of skin. Medical treatment include narrow band UVB and excimer laser treatment where instrumentation is available. Surgical treatment includes relocating skin grafts from pigmented to depigmented areas. Alternatively, melanocytes are isolated from the skin and transferred to the affected skin. The efficacy of all of the above treatments is limited as not every individual responds to these treatments, or repigmentation does not last. Newly introduced pigment cells remain vulnerable to autoimmune removal and repeated loss of pigmentation

### SUMMARY

In one aspect, described herein is a method of treating of treating vitiligo in a subject in need thereof, comprising administering to the subject an regulatory T cell engineered to express a chimeric antigen receptor (CAR) that specifically binds ganglioside D3. In some embodiments, the cells are autologous. In some embodiments, the administration of the cells reduces depigmentation in the skin of the subject. For example, in some embodiments, the administration of the cells results in a 50% decrease in depigmentation over the treatment period compared to subjects not receiving the cells. In some embodiments, the administration of the cells results in at least a 2.5 fold increase in IL-10 expression by CAR Treg in response to relevant target cells as measured by ELISA. In some embodiments, the administration of cells results in a decrease in cytotoxicity over 24 hrs compared to target cells plus cytotoxic T cells alone.

The cells can be administered intravenously or subcutaneous injection.

In some embodiments, the subject is also suffering from alopecia, hypothyroid disease or other vitiligo-associated autoimmune diseases.

### BRIEF DESCRIPTION OF THE FIGURES

FIG. 1 shows a schematic of a CAR construct. DNA codes for a protein containing variable regions of a single chain antibody fused to a CD8 hinge and CD28 extracellular, transmembrane and intracellular signaling region, and TCR zeta cytoplasmic domain. This is introduced into regulatory T cells, and the resulting transgenic CAR Treg can effectively inhibit cytotoxic T cell activity upon on contact upon an encounter with target antigen. The GD3 target molecule, which is convincingly expressed in perilesional vitiligo skin, primarily by melanocytes.

FIG. 2 outlines the protocol used to generate Treg in vitro as described in the Example.

FIGS. 3A and 3B are plots showing the high transduction efficiencies are observed for Tregs expressing the GD3 CAR. CD4+FoxP3+ cells, polarized from naïve CD4+ T cells, were transduced using a GD3 CAR-encoding construct. (A) The gating strategy consists of a time gate followed by sequentially gating on lymphocytes, single cells, and live cells. (B) Eighty-six percent of total CD4+ T cells were successfully transduced to express the GD3 CAR construct and 67% of that population express FoxP3+.

FIG. 4 is a graph showing that CAR Tregs but not untransduced Tregs overexpress IL-10 by about 3-fold in presence of GD3-expressing target cells.

FIG. 5 is a graph showing melanocyte viability over time, showing reduced target cell death in presence of CAR Tregs.

FIG. 6 shows that reduced depigmentation was observed following adoptive transfer of GD3 CAR Tregs to vitiligo-prone h3T^{+/-}A2^{+/+} mice.

FIG. 7 is a graph showing that Trp1+ melanocytes were maintained in the presence of Tregs.

FIG. 8 are graphs showing that CD3/FoxP3 Tregs were more abundant in the skin of mice after adoptive cell transfer.

FIG. 9 is a graph showing the average percent depigmentation in mice treated with the CAR CD3 Tregs and untreated mice over time.

FIGS. 10A-10C show that GD3 CAR Tregs generate immunosuppressive cytokines in presence of activated T cells, Cytokines were measured in supernatants from cocultures of melanocyte targets and HLA-A2-restricted Teffs, in presence and absence of untransduced or CAR-transduced Tregs. Cytokine concentrations for each coculture, measured in triplicate cocultures, are shown for (A) IFN-γ, (B) TNF-α, (C) IL-4, and (D) IL-10. Statistical analysis was performed by a one-way ANOVA test followed by Tukey's post-hoc test for multiple comparisons. *p<0.05; **p<0.01; ***p<0.001; ****p<0.0001.

FIG. 11A shows that GD3 CAR Tregs provide melanocytes with superior protection from T cell-mediated cytotoxicity in vitro The immunosuppressive ability of GD3 CAR Tregs and untransduced Tregs was compared in vitro and the viability of HLA-A2+ human melanocytes (targets) in the presence or absence of murine Teffs and Tregs (1:10:1) is represented over time.

FIGS. 12A-12C are graphs showing the individual mouse depigmentation values over time support the treatment effects of CAR Tregs. Dorsal depigmentation, represented as the change from baseline over time, is shown for each individual mouse from the (FIG. 12A) vehicle treated (n=12), (FIG. 12B) untransduced Treg (n=11), and (FIG. 12C) GD3 CAR Treg (n=11) treated groups. Respective ventral depigmentation values for (FIG. 12D) vehicle, (FIG. 12E) untransduced Tregs and (FIG. 12F) CAR Treg are also presented.

FIGS. 13A-13D show that GD3 CAR Tregs provide significant protection from depigmentation in vitiligo-prone mice. (FIG. 12A) Experimental outline showing vitiligo prone, h3T-A2, mice treated with vehicle alone (n=12), or by adoptive transfer of untransduced Tregs (n=11) or GD3 CAR Tregs (n=11). Adoptive transfer started at 5 weeks of age and continued biweekly until 11 weeks of age. Depigmentation was measured weekly from 5-15 weeks of age. (FIG. 13B) Representative dorsal and ventral scans of mice from the HBSS vehicle, untransduced Treg, and GD3 CAR Treg treated groups. (FIG. 13C) Depigmentation quantified on dorsal and (FIG. 13D) ventral sides throughout the experiment. The Wilcoxon rank sum (WRS) test was used to compare the time-adjusted AUC among groups. Arrows: treatment times. *p<0.05; **p<0.01.

FIG. 14A-14C show that melanocytes are protected from h3T cytotoxic T cells in the presence of GD3 reactive CAR Tregs. FIG. 14A is a bar graph showing the quantification of TRP-1+ cells/mm² in melanocytes transduced with vehicle (HBSS), untransduced Tregs, GD3 CAR transduced Tregs. FIG. 14B is a bar graph showing the quantification of CD3+ cells/mm² in melanocytes transduced with vehicle (HBSS), untransduced Tregs, GD3 CAR transduced Tregs. FIG. 14C is a bar graph showing the quantification of CD3+FoxP3+ cells/mm² in melanocytes transduced with vehicle (HBSS), untransduced Tregs, GD3 CAR transduced Tregs (Statistical analysis was performed by non-parametric t tests. *p<0.05, **p<0.001 (Scale bar=20 μm).

FIG. 15 is a bar graph that shows that Treg transfusion helps maintain GD3 expressing cells in h3T-A2 vitiligo mouse skin. Quantification of GD3 expressing cells from h3T-A2 mouse skin at end point (mean±SD) is compared across recipients of vehicle treatment, adoptive transfer by untransduced Tregs, or by GD3 CAR Tregs (n=3 per group). Statistical significance was determined by one-way ANOVA followed by a Tukey post-test to correct for multiple comparisons *p<0.05; **p<0.01.

FIG. 16 is a schematic presentation of adoptive transfer of CAR Tregs in vitiligo. Autoimmune melanocyte destruction is mediated by cytotoxic T cells, which are activated via self-antigens secreted by stressed melanocytes. Elevated IL-17 promotes inflammatory environment in the skin. Infusion of GD3-specific CAR Tregs potentially migrate towards cognate antigen at the site of autoimmune activity, and suppress cytotoxic T cells via bystander effect, and provide a local immune tolerance in vitiligo skin.

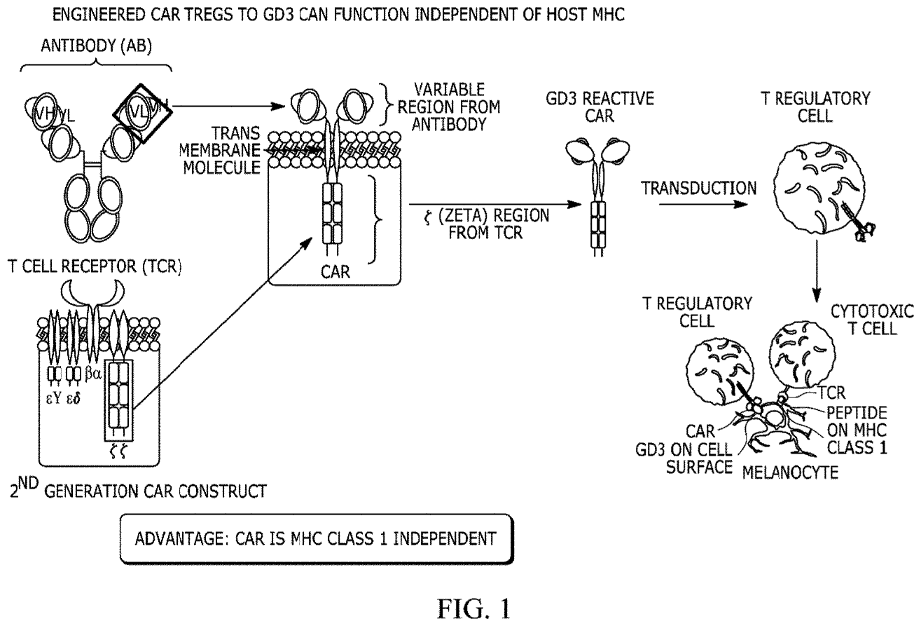

### DETAILED DESCRIPTION

The present disclosure is directed to the use of T cells engineered with a chimeric antigen receptor (CAR) designed to target Ganglioside D3 (GD3) to treat the autoimmune disease vitiligo. GD3 is a tumor-associated antigen otherwise found in melanoma and neuroendocrine tumors; normal expression is largely restricted to neuronal cells in the brain during development.

Regulatory T cells (Tregs) are crucial to inducing peripheral self-tolerance in vitiligo. The number of immunosuppressive Tregs among T cell infiltrates in vitiligo lesions is greatly reduced compared to healthy skin, suggesting that restoring cutaneous Tregs might protect against depigmentation. Antigen-specific CAR Tregs generated against GD3, a melanocyte antigen which is overexpressed in the lesional epidermis, secrete significantly more IL-10 compared to untransduced Tregs to mediate suppressive function in vitro.

As described herein in the Example, to generate GD3-specific CAR Tregs for adoptive transfer, naïve CD4+ T cells originated from FoxP3 eGFP reporter mice were polarized to CD4+FoxP3+ Tregs in the presence of TGF-β. To obtain sufficient amount of Tregs, anti-CD3/CD28 T cell activator beads and high concentration of IL-2 were included in the culture, which enhanced Treg numbers by 8-fold over 5 days.

Subsequently, the suppressive activity of GD3-specific CAR Tregs versus untransduced Tregs was assessed. GD3 CAR Tregs (n=11) and untransduced Tregs (n=11) were adoptively transferred to h3TA2 recipient mice biweekly for four rounds when mice were 5, 7, 9, and 11 weeks old. An untreated group (n=12) was maintained for comparison. As shown in the Example, in comparison to GD3-targeted CAR Tregs, mice treated with untransduced Tregs exhibited a 3-fold increase (p=0.0404) in average depigmentation, while untreated control mice experienced a 3-fold increase in depigmentation over 10 weeks, indicating that antigen-specific CAR Tregs maintained prolonged immunosuppression in vitiligo-prone mice.

### Chimeric Antigen Receptor

A chimeric antigen receptor (CAR) is designed for a T cell and is a chimera of a signaling domain of the T cell receptor (TCR) complex and an antigen-recognizing domain (e.g., a single chain fragment (scFv) of an antibody or other antibody fragment) (Enblad et al., Human Gene Therapy. 2015; 26(8):498-505). A T cell that expresses a CAR is referred to as a CAR T cell. CARs have the ability to redirect T cell specificity and reactivity toward a selected target in a non-MHC-restricted manner. The non-MHC-restricted antigen recognition gives T cells expressing CARs the ability to recognize an antigen independent of antigen processing, thus bypassing a major mechanism of target cell escape. When expressed in T cells, CARs advantageously do not dimerize with endogenous T cell receptor (TCR) alpha and beta chains.

There are four generations of CARs, each of which contains different components. First generation CARs join an antibody-derived scFv to the CD3zeta (ζ or z) intracellular signaling domain of the T cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains may include, e.g., a combination of CD3ζ, CD27, CD28, 4-1BB, ICOS, or OX40. CARs, in some embodiments, contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3ζ and/or co-stimulatory molecules (Maude et al., Blood. 2015; 125(26):4017-4023; Kakarla and Gottschalk, Cancer J. 2014; 20(2):151-155). Fourth-generation CARs contain three costimulatory domains. In some embodiments, the CAR used in the methods described herein is a first-generation CAR. In some embodiments, the CAR used in the methods described herein is a second-generation CAR. In some embodiments, the CAR used in the methods described herein is a third-generation CAR. In some embodiments, the CAR used in the methods described is a fourth-generation CAR.

CARs typically differ in their functional properties. The CD3ζ signaling domain of the T cell receptor, when engaged, will activate and induce proliferation of T cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T cells. Similar anti-target cell effects are observed in vitro with CD28 or 4-1BB CARs, but preclinical in vivo studies suggest that 4-1BB CARs may produce superior proliferation and/or persistence. Third generation CARs combine multiple signaling domains (costimulatory) to augment potency.

The extracellular domain is the region of the CAR that is exposed to the extracellular fluid and, in some embodiments, includes an antigen binding domain, and optionally a signal peptide, a spacer domain, and/or a hinge domain. In some embodiments, the antigen binding domain is a single-chain variable fragment (scFv) that include the VL and VH of an immunoglobulin connected with a short linker peptide. The linker, in some embodiments, includes hydrophilic residues with stretches of glycine and serine for flexibility, optionally also with stretches of glutamate and lysine for added solubility. A single-chain variable fragment (scFv) is a fusion protein of the variable regions of the heavy chain (VH) and light chain (VL) of immunoglobulins, connected with a short linker peptide of ten to about 25 amino acids. The linker can either connect the N-terminus of the VH with the C-terminus of the VL, or vice versa. This protein retains the specificity of the original immunoglobulin, despite removal of the constant regions and the introduction of the linker.

In some or any embodiments, the scFv binds to human GD3 (Genbank Accession No. CAA54891.1), or a naturally occurring variant thereof, with an affinity (Kd) of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹², or ranging from 1×10⁻⁹ to 1×10⁻¹⁰, or ranging from 1×10⁻¹² to about 1×10⁻¹³. Affinity is determined using a variety of techniques, examples of which include an affinity ELISA assay and a surface plasmon resonance (BIAcore) assay.

Non-limiting examples of VH and VL protein sequences that may be used to create an anti-gangioloside D3 (GD3) scFv may include the VH and VL regions of the anti-GD3 antibody disclosed in Houghton, A. N. et al, Proc. Natl. Acad. Sci. USA. 82:1242-1246, 1985; the VH and VL regions disclosed in SEQ ID NOs: 55 and 56, respectively, set forth in International Publication No. WO 2001/023432.

In some embodiments, the GD3 scFv comprises a VH amino acid sequence set forth in SEQ ID NO: 1 and a VL amino acid sequence set forth in SEQ ID NO: 2. See U.S. Patent Publication No. 2007/0031438, the disclosure of which is incorporated herein by reference in its entirety.

In some embodiments, the anti-GD3 scFv is humanized. In other embodiments, the anti-GD3scFv is fully human. In yet other embodiments, the anti-CD3 scFv is a chimera (e.g., of mouse and human sequence).

In some embodiments, the CAR comprises the VH and VL regions of the anti-GD3 monoclonal antibody (mAb) MB3.6 described in Lo et al., Clin Cancer Res., 16:2769-2780, 2010, and Cheresh et al., Proc Natl Acad Sci USA, 82:5155-5159, 1985, the disclosures of which are incorporated by reference in their entireties.

A signal peptide can enhance the cellular export and membrane localization of the CAR by host cells.

In some embodiments, a spacer domain or hinge domain is located between an extracellular domain (comprising the antigen binding domain) and a transmembrane domain of a CAR, or between a cytoplasmic domain and a transmembrane domain of the CAR. A spacer domain is any oligopeptide or polypeptide that functions to link the transmembrane domain to the extracellular domain and/or the cytoplasmic domain in the polypeptide chain. A hinge domain is any oligopeptide or polypeptide that functions to provide flexibility to the CAR, or domains thereof, or to prevent steric hindrance of the CAR, or domains thereof. In some embodiments, a spacer domain or a hinge domain may comprise up to 300 amino acids (e.g., 10 to 100 amino acids, or 5 to 20 amino acids). In some embodiments, one or more spacer domain(s) may be included in other regions of a CAR. In some embodiments, the hinge domain is a CD8 hinge domain. Other hinge domains may be used.

The transmembrane domain of the CAR is, in various embodiments, a hydrophobic alpha helix that spans the membrane. The transmembrane domain provides stability of the CAR. In some embodiments, the transmembrane domain of a CAR as provided herein is a CD8 transmembrane domain. In other embodiments, the transmembrane domain is a CD28 transmembrane domain. In yet other embodiments, the transmembrane domain is a chimera of a CD8 and CD28 transmembrane domain. Other transmembrane domains may be used as provided herein. Other transmembrane domains may be used.

The endodomain is the functional end of the receptor. Following antigen recognition, receptors cluster and a signal is transmitted to the cell. The most commonly used endodomain component is CD3-zeta, which contains three (3) immunoreceptor tyrosine-based activation motif (ITAM)s. This transmits an activation signal to the T cell after the antigen is bound. In many cases, CD3-zeta may not provide a fully competent activation signal and, thus, a co-stimulatory signaling is used. For example, CD28 and/or 4-1BB may be used with CD3-zeta (CD3ζ) to transmit a proliferative/survival signal. Thus, in some embodiments, the co-stimulatory molecule of a CAR as provided herein is a CD28 co-stimulatory molecule. In other embodiments, the co-stimulatory molecule is a 4-1BB co-stimulatory molecule. In some embodiments, a CAR includes CD3ζ and CD28. In other embodiments, a CAR includes CD3-zeta and 4-1BB. In still other embodiments, a CAR includes CD3ζ, CD28, and 4-1BB.

### CAR T Cells

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some embodiments, an isolated population of T cells is used. A specific subpopulation of T cells, expressing one or more of the following cell surface markers: CD3, CD4, CD45, can be further isolated by positive or negative selection techniques.

To achieve sufficient therapeutic doses of T cell populations, T cells are often subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to being contacted with a CAR composition.

In some embodiments, T cells are activated and expanded for about 4 hours, about 6 hours, about 12 hours, about 18 hours, about 24 hours, about 36 hours, about 48 hours, about 60 hours, or about 72 hours prior to being contacted with a nucleic acid encoding the CAR construct.

Viral and non-viral based gene transfer methods can be used to introduce nucleic acids encoding the CAR construct to cells (e.g., T cells). Viral vector delivery systems include DNA and RNA viruses (e.g., lentiviral vector or retroviral vector), which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

In some embodiments, the nucleic acid encoding a CAR construct can be delivered to a cell using a lentivirus or a retrovirus.

Successfully transduced or transfected cells can then be re-sorted by a Treg sorting kit combined with antibodies directed to the CAR to achieve a highly Treg skewed, CAR expressing population.

The cells can be incubated in cell medium in a culture apparatus for a period of time or until the cells reach a sufficient cell density for optimal passage before passing the cells to another culture apparatus by rapid expansion. The culturing apparatus can be of any culture apparatus commonly used for culturing cells in vitro. The cell medium may be replaced during the culture of the cells at any time. Preferably, the cell medium is replaced about every 2 to 3 days. The cells are then harvested from the culture apparatus whereupon the cells can be used immediately or cryopreserved to be stored for use at a later time. In one embodiment, the expanded cells are cryopreserved prior to administration in amounts suitable for a single treatment.

Therapeutic Method

In one aspect, described herein is a method of treating an autoimmune disease in a subject in need thereof comprising administering to the subject a regulatory T cell engineered to express a chimeric antigen receptor (CAR) that specifically binds ganglioside D3. In some embodiments, the autoimmune disease is vitiligo.

A subject may be any subject for whom diagnosis, treatment, or therapy is desired. In some embodiments, the subject is a mammal. In some embodiments, the subject is a human.

In some embodiments, the CAR T cells are autologous; that is, the T cells are obtained or isolated from a subject and administered to the same subject, i.e., the donor and recipient are the same. In some embodiments, syngeneic cell populations may be used, such as those obtained from genetically identical donors (e.g., identical twins).

The CAR T cells administered according to the methods described herein do not induce toxicity in the subject. In some embodiments, an engineered T cell population being administered does not trigger complement mediated lysis, or does not stimulate antibody-dependent cell mediated cytotoxicity (ADCC).

An effective amount of CAR T cells are administered, e.g., an amount which prevents or alleviates at least one or more signs or symptoms of a medical condition (e.g., depigmentation). “An effective amount” also relates to a sufficient amount of a composition comprising the CAR T cells to provide the desired effect, e.g., to treat a subject having a medical condition. An “effective amount” also includes an amount of therapeutic sufficient to prevent or delay the development of a symptom of the disease, alter the course of a symptom of the disease (for example, but not limited to, slow the progression of a symptom of the disease), or reverse a symptom of the disease.

For use in the various aspects described herein, an effective amount of cells (e.g., engineered CAR T cells) comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, at least 1×10⁷ cells, at least 2×10⁷ cells, at least 3×10⁷ cells, at least 4×10⁷ cells, at least 5×10⁷ cells, at least 6×10⁷ cells, at least 7×10⁷ cells, at least 8×10⁷ cells, at least 9×10⁷ cells, at least 1×10⁸ cells, at least 2×10⁸ cells, at least 3×10⁸ cells, at least 4×10⁸ cells, at least 5×10⁸ cells, at least 6×10⁸ cells, at least 7×10⁸ cells, at least 8×10⁸ cells, at least 9×10⁸ cells, at least 1×10⁹ cells, at least 2×10⁹ cells, at least 3×10⁹ cells, at least 4×10⁹ cells, at least 5×10⁹ cells, at least 6×10⁹ cells, at least 7×10⁹ cells, at least 8×10⁹ cells, at least 9×10⁹ cells, or multiples thereof. In some embodiments, an effective amount of cells comprises at least 1×10⁵ cells. In some embodiments, an effective amount of cells comprises at least 3×10⁸ cells. In some embodiments, an effective amount of cells comprises an amount ranging from about 1×10⁵ cells to about 3×10⁸ cells. In some embodiments, the effective amount of cells comprising an amount ranging from about 1×10⁵ cells to about 9×10⁹ cells.

In some embodiments, an effective amount of cells (e.g., engineered CAR T cells) is administered as a number of cells per kg of the subject receiving treatment. For example, in some embodiments, the effective amount of cells ranges from at least about 1×10² cells/kg to about at least 9×10⁶ (or about 1×10² cells/kg cells/kg to about 1×10⁶ cells/kg, or about at 4×10⁵ cells/kg to about 6×10⁵ cells/kg, or about 5×10⁵ cells/kg to about 9×10⁵ cells/kg, or about 4×10⁶ cells/kg to about 6×10⁶ cells/kg, or about 5×10⁶ cells/kg to about 9×10⁶ cells/kg). In some embodiments, the effective amount of cells comprises at least 1×10² cells/kg, at least 5×10² cells/kg, at least 1×10³ cells/kg, at least 5×10³ cells/kg, at least 1×10⁴ cells/kg, at least 5×10⁴ cells/kg, at least 1×10⁵ cells/kg, at least 2×10⁵ cells/kg, at least 3×10⁵ cells/kg, at least 4×10⁵ cells/kg, at least 5×10⁵ cells/kg, at least 6×10⁵ cells/kg, at least 7×10⁵ cells/kg, at least 8×10⁵ cells/kg or at least 9×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg or at least 9×10⁶ cells/kg. In some embodiments, the amount of cells comprises about 1×10⁵ cells/kg of subject.

The cells are derived from one or more donors, or are obtained from an autologous source. In some examples described herein, the cells are expanded in culture prior to administration to a subject in need thereof.

Modes of administration include injection, infusion, or instillation. Injection includes, without limitation, intravenous, intramuscular, intra-arterial, intrathecal, intraventricular, intracapsular, intraorbital, periorbital, intracardiac, intradermal, intraperitoneal, transtracheal, subcutaneous, subcuticular, intraarticular, sub capsular, subarachnoid, intraspinal, intracerebro spinal, and intrasternal injection. In some embodiments, engineered CAR T cells are administered systemically, which refers to the administration of the cells other than directly into a target site, tissue, or organ, such that it enters, instead, the subject's circulatory system. In some embodiments, the route of administration is intravenous. In some embodiments, the route of administration is subcutaneous injection.

In various aspects, the method described herein improves one or more signs or symptoms of the autoimmune disease in the subject. Any level of improvement is contemplated. In the context of vitiligo, the method optionally reduces depigmentation in the subject. Alternatively, the method optionally slows the onset of depigmentation (or slows the worsening of depigmentation) at one or more sites on the body. Methods of measuring indicators of other autoimmune disease are known to those of skill in the art and/or described herein. “Treatment” includes any treatment of the disease in subject and includes: (1) inhibiting the disease, e.g., arresting, or slowing the progression of symptoms; (2) relieving the disease, e.g., causing regression of symptoms; and/or (3) preventing or reducing the likelihood of the development of symptoms.

### EXAMPLES

Materials and Methods

Cell Culture and Reagents:

Mouse naive CD4+ T cells and CD4+FoxP3+ Tregs from a FoxP3 reporter mouse were cultured in T cell media (RPMI media supplied with 10% FBS, 1× Non-essential amino acids (Corning, Cat #25-025-CI), 50 U/ml Penicillin-Streptomycin (Thermo Fisher Scientific, Cat #15140122), 1 mM Sodium Pyruvate (Gibco, Cat #11360-070), 10 mM HEPES (Gibco, Cat #15630-080), 50 μM β-Mercaptoethanol (Sigma, Cat #M-7522)). Human melanocytes (HLA-A2 positive #Mf0887, P6; HLA-A2 negative #Ms18001, P7) were cultured in human melanocyte media (Human Melanocyte Growth Supplement-2, PMA-free (HMGS-2) (cat #50165); Medium 254 (cat #254500); 10 mM L-Glutamine (cat #A2916801); 1× Antibiotic-antimycotic (cat #15240062). Rabbit anti-GD3 CAR sera and stable GD3 CAR virus producing cells were described in Lo et al., Clin Cancer Res., 16:2769-2780, 2010. PG10 stable GD3 CAR virus producing cells were maintained in T cell media for virus production.

Car Construct:

The second-generation (Tandem) chimeric receptor (sFv-CD28/TCRζ) was created as sFv (243 amino acids)-CD8α hinge (46 amino acids)-nucleotides 334 to 660 (109 amino acids) of CD28 [a portion of extracellular domain (44 amino acids), transmembrane (24 amino acids), and intracellular domain (41 amino acids)]-ζ chain (intracellular domain, 112 amino acids) by replacing the anti-carcinoembryonic antigen (CEA) sFv in the second-generation anti-CEA CAR with MB3.6 sFv via NotI and HindIII sites. See in Lo et al., Clin Cancer Res., 16:2769-2780, 2010, the disclosure of which is incorporated herein in its entirety.

Isolation of Naive CD4+ T Cells and Polarization to CD4+ FoxP3+ Tregs In Vitro:

Naive mouse CD4+ T cells were isolated from spleen of 8-10 weeks FoxP3 eGFP reporter mouse (Jackson Laboratories, stock No. 006772) using EasySep Mouse naive CD4+ T cell Isolation Kit (STEMCELL Technologies, Cat #19765) following protocol provided by manufacturer. These mice co-express eGFP, which is restricted to the T cell lineage, primarily to the CD4⁺ T cell population. Naive CD4+ T cells were polarized to CD4+FoxP3+ using 30 ng/ml human TGF-β in the presence of Dynabeads™ Mouse T-Activator CD3/CD28 (Thermo Fisher Scientific, Cat #11452 D) with a 1:1 beads to cell ratio and 300 IU/ml rhIL-2 for 5 days. Human TGF-β was used to polarize murine Tregs as mouse and human TGF-β share 99% sequence homology with high cross-species reactivity (Abnaof et al. 2014, Tsang et al. 1995). Human IL-2 was used as human and mouse IL-2 share 57% of homology, and human IL-2 efficiently stimulates mouse IL-2 receptor, whereas mouse IL-2 do not elicit efficient binding to human IL-2 receptors (Arkin et al. 2003, Arenas-Ramirez, Woytschak, and Boyman 2015).

Generation of GD3 CAR Transduced Mouse Tregs:

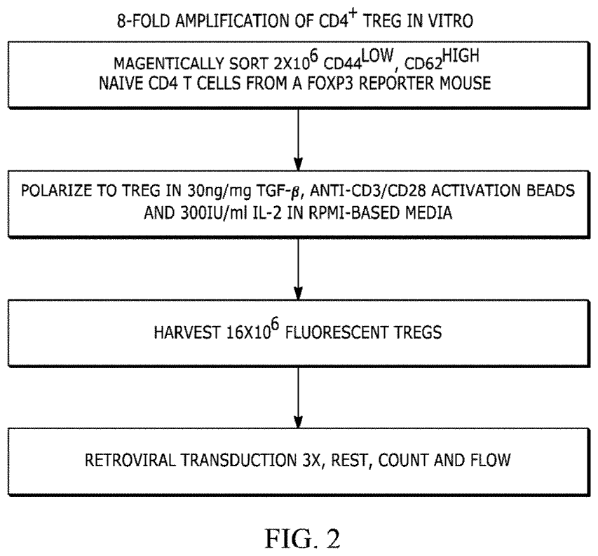

24 well non-tissue culture plate were coated with 10 μg/ml retronectin (Takara, Cat #50-444-032) for 2 hours at room temperature. MFG retroviral vector based second generation CAR construct (sFv-CD28/TCRζ) reactive GD3 was generated (Lo et al. 2010a). One mL supernatant out of 10 ml GD3 CAR VPC culture medium (80% confluent in 10 cm diameter plate) was transferred to a retronectin coated plate and centrifuged at 2000 g for 2 hours. After centrifugation, supernatant was carefully removed and 1 million activated CD4+FoxP3+ Tregs were transferred to retronectin coated plate with additional 1 ml viral supernatant, 5 μg/ml Protamine sulfate and 300 IU/mL rhIL2. The retronectin plate with activated T cells was centrifuged at 1000 g for 1 hour, followed by 4-hour incubation at 37° C., adding fresh with complete T cell culture medium with Dynabeads™ Mouse T-Activator CD3/CD28 beads 1:1 bead to cell ratio and 300 IU/ml rhIL-2. Second transduction followed the same protocol as described above to increase transduction efficiency. Transduced Tregs were reactivated with CD3/CD28 beads at 1:1 beads to cell ratio in the presence of 30 ng/ml human TGF-β and 300 IU/ml rhIL-2 and recovered for 2 days before flow analysis. See FIG. 2.

Flow Cytometry:

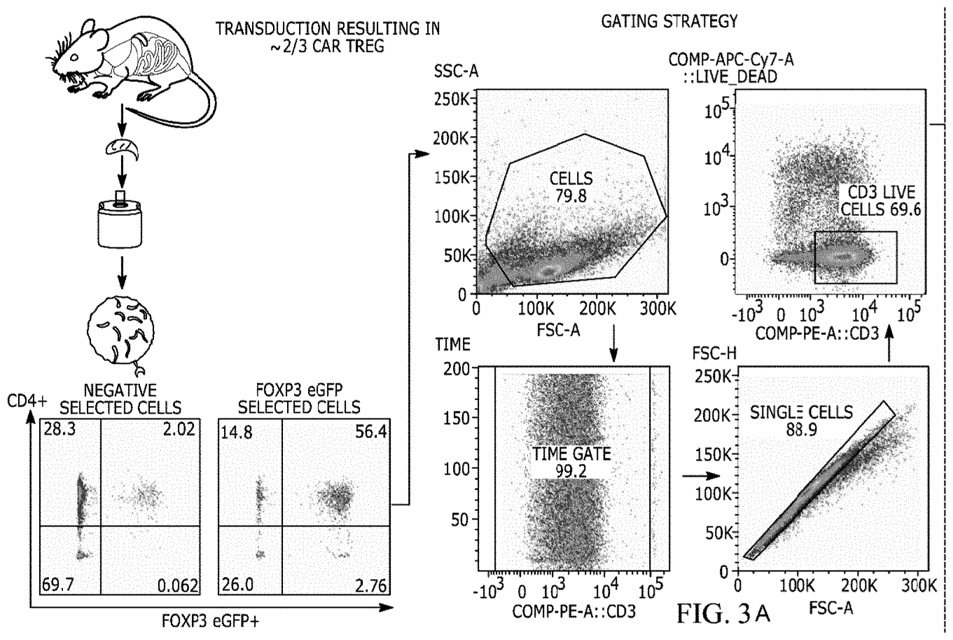

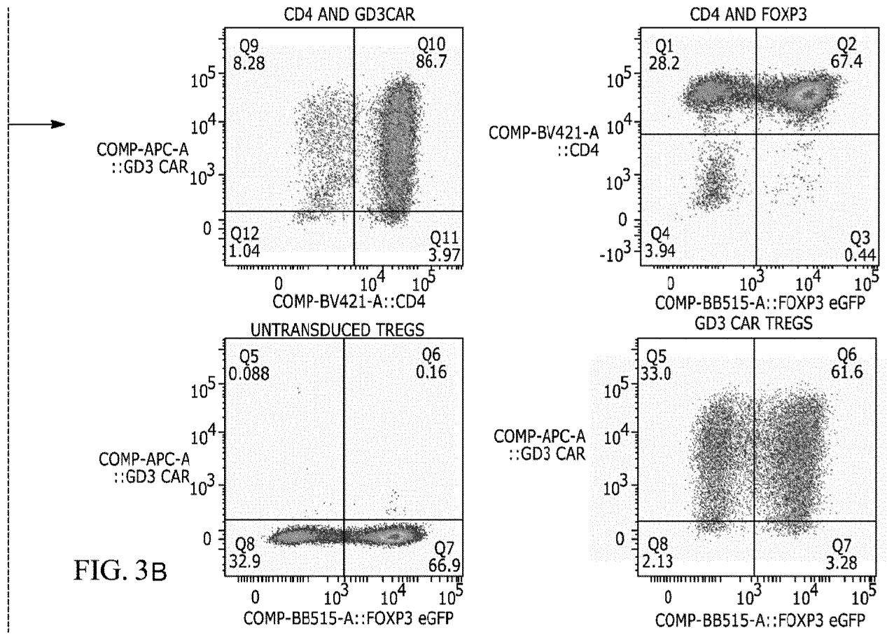

Prior to surface staining, cells were incubated with mouse Fc Block (Biolegend) and LIVE/DEAD Fixable Near IR Dead Cell dye (Thermo Fisher Scientific) according to manufacturer's instructions. Surface staining of directly labeled antibodies included anti-CD3 BUV395 (BD Biosciences, 145-2C11, cat #563565), CD4 BV421 (BioLegend, GK1.5, Cat #100443), FoxP3 eGFP BB515, and unlabeled anti-GD3 CAR rabbit sera (secondary antibody-anti-rabbit APC (Invitrogen, A10931). Stained cells were run using a BD FACSymphony flow cytometer, and analyzed using FlowJo v10.3.0 (FlowJo LLC, OR, USA). See FIG. 3.

In Vitro Co-Culture Experiments:

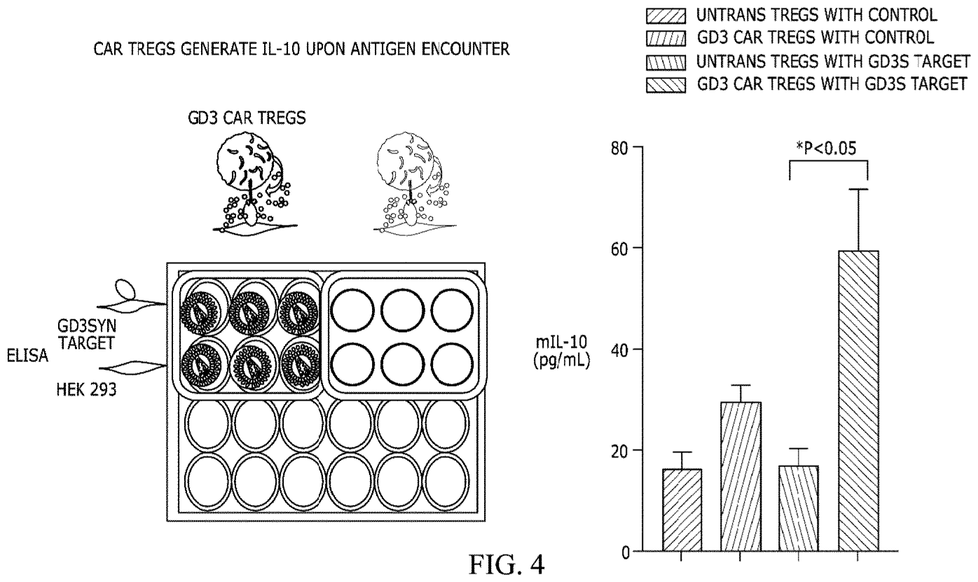

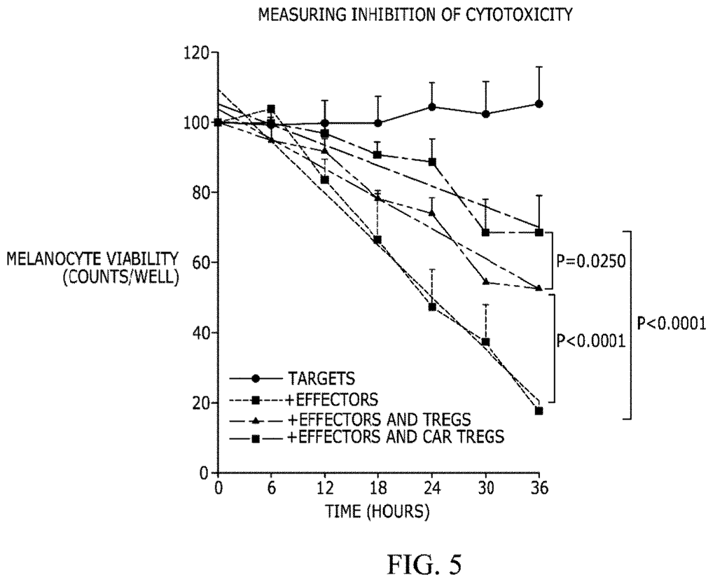

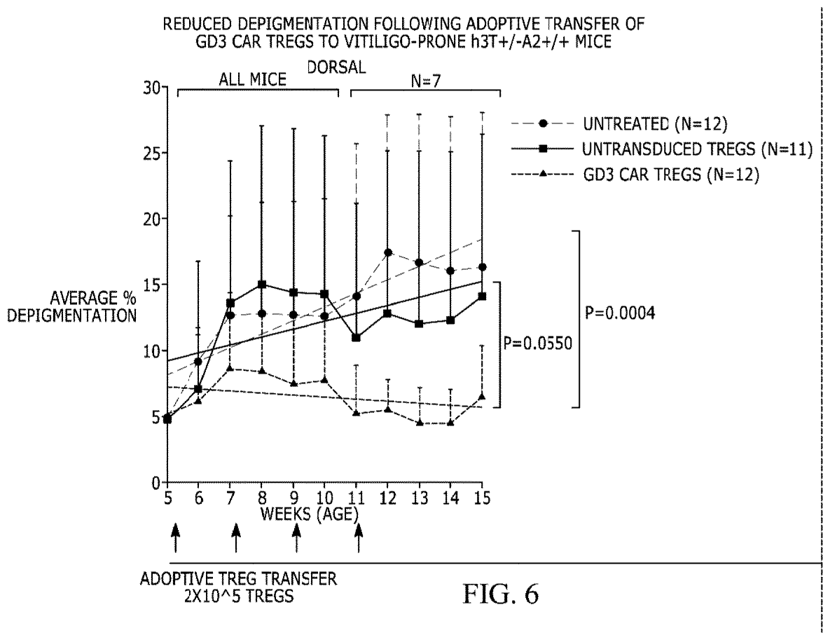

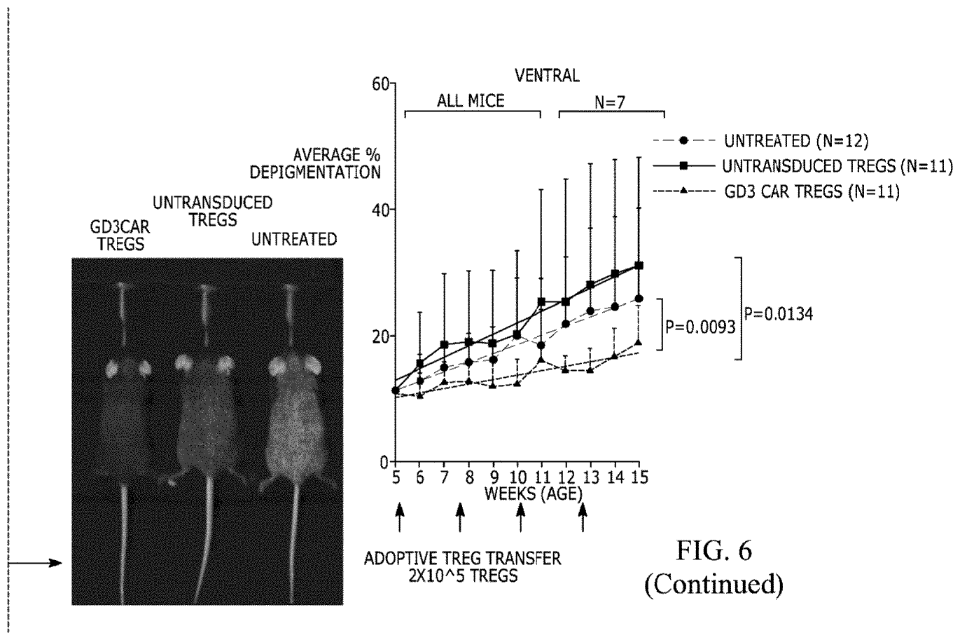

HLA-A2⁺ melanocytes were identified by immunofluorescence staining using FITC-labeled BB7.2 to human HLA-A2 prior to in vitro co-culture experiments. Human HLA-A2⁺ neonatal foreskin melanocytes (Mf0887, P6) and HLA-A2-abdominoplastic skin melanocytes (Ms18001, P6) melanocytes were seeded together with human tyrosine reactive effector T cells (h3T T cells) and untransduced/GD3 CAR-transduced suppressor Tregs at 10:1:1 effector to target to suppressor ratio for 36 hours. Teff:Tregs ratio was used to mimic the natural occurrence of the T cell subsets as Tregs comprise 5-10% of the total T cell population. Co-cultures were seeded in triplicates and incubated using IncuCyte® Caspase-3/7 Red Apoptosis Assay Reagent (Cat. No. 4704). Images were taken every three hours in triplicates using IncuCyte® live-cell analysis system. See FIG. 5. Supernatant was saved for mouse IFNγ and IL-10 ELISA assay (ab) following manufacturer's protocol. See FIG. 4. Cytotoxicity was examined by measuring the remaining cells relative to targets only control cells using Photoshop (data not shown).

Cytokine Analysis:

Included in cytokine analysis were supernatants from in vitro suppression assays (IncuCyte experiments), collected 36 hours post-co-culture, and serum samples from HBSS vehicle (n=11), untransduced (n=10) and GD3 CAR Tregs (n=9) treated mouse groups. Detection of murine IFN-γ, TNF-α, IL-4 and IL-10 was performed by using a V-Plex Proinflammatory Panel 1 Mouse kit (Meso Scale Diagnostics, LLC) according to manufacturer's instructions. Data were acquired on a Synergy HT reader (Biotek) equipped with Gen5 v1.08 (Biotek) and analyzed using Prism version 8.3.0 (GraphPad Software).

Statistical Analysis:

Statistical analysis was performed using GraphPad Prism 8.0 software (GraphPad) and R-software. Data are presented as bars and dot plots with mean values±standard deviation. The data were evaluated by one-way ANOVA analysis of variance accounting for different variances across the treatment groups, with post-hoc Tukey-Kramer comparisons. To determine statistical significance for immunosuppression in vitro, two-way ANOVAs were used with aligned rank transformation followed by multiple pairwise comparison testing using Tukey approach. For depigmentation, the time-adjusted AUC, representing change in depigmentation from treatment initiation, was calculated using the trapezoidal rule. No imputation was done for missing data, and the AUC for each mouse was divided by the total number of weeks of available data minus 1. The Wilcoxon rank sum (WRS) test was used to compare the time-adjusted AUC among groups. Statistical significance is represented as *p<0.05, ** p<0.01, *** p<0.001 or **** p<0.0001.

### Example 1—Adoptive Transfer of Tregs in h3TA2 Mice

Transgenic recipient mice with TCR reactive to the human tyrosinase 368-376 (YMDTMSQV) epitope, h3TA2 (Mehrotra et al., 2012; Chatterjee et al. 2014), were maintained under protocols approved by Northwestern University's Institutional Animal Care and Use Committee (IACUC) following the institutional guidelines. Mice were administered retro-orbitally with 2×10⁵ untransduced Tregs/per animal n=11 (6♂, 5♀) or 2×10⁵ GD3 CAR Tregs/per animal n=11 (6♂, 5♀), every two weeks, four times, starting at 5-week age. The number of adoptively transferred Tregs was identified to enable a comparison to previous studies (Chatterjee et al. 2014), where the 2×10⁵ polyclonal Tregs controlled depigmentation in h3TA2 mouse model between 3-9 weeks old mice. Recombinant human IL-2 also was administered at 3000 IU/per animal, 3 times a week. A cohort was left untreated n=12 (6♂, 6♀). The experiment was maintained until week 15, and the experiment was terminated. Skin biopsies, spleen, brain, ileum, lymph nodes were maintained in OCT, and serum was stored for cytokine analysis.

Depigmentation Measurements:

From 5 weeks to 15 weeks of age, mice were scanned every week by flatbed scanning (Hewlett-Packard, Palo Alto, Calif.) under isoflurane anesthesia. Using Adobe Photoshop software (Adobe Systems, San Jose, Calif.) luminosity was measured (Denman et al., 2008) for the ventral and dorsal part of mice, with a fully pigmented skin representing 0% depigmentation and a fully depigmented skin representing 100% depigmentation. Depigmentation was graphed over time and slopes were calculated using Prism software (GraphPad, San Diego, Calif.) and compared among the untransduced, GD3 CAR transduced, and untreated groups.

Immunohistology:

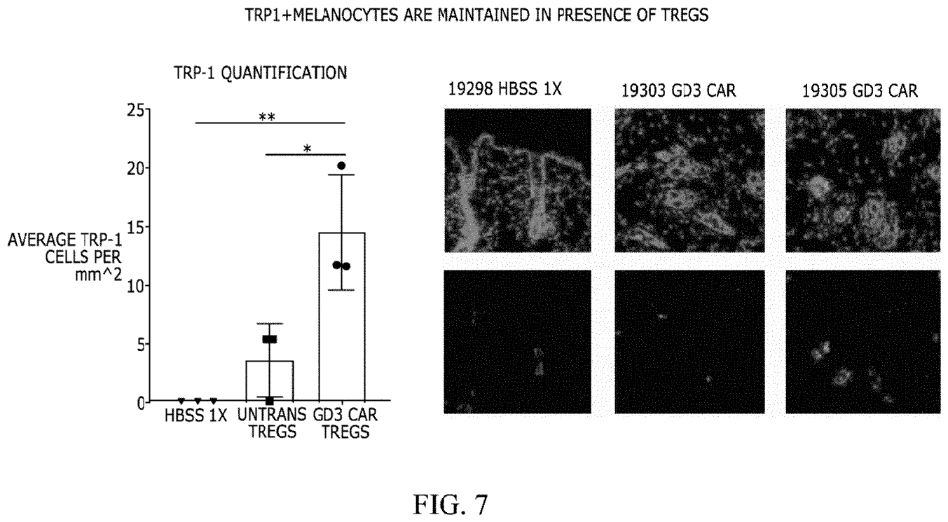

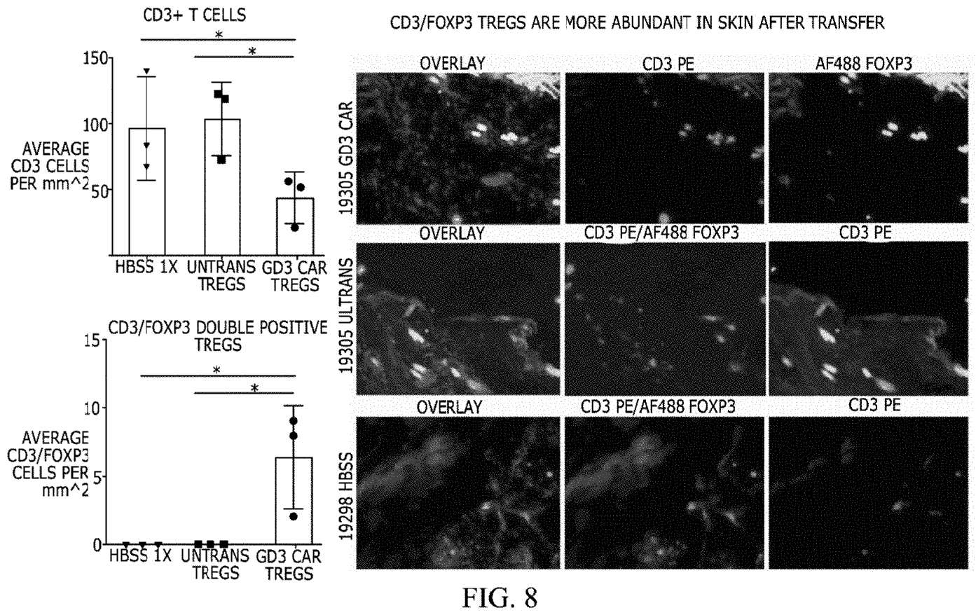

Mouse and human skin samples were frozen using Optimal Cutting Temperature Compound in dry ice (Sakura Finetek, Torrance, Calif.). Tissues were cryosectioned at 8 μm using (Leica, Wetzlar, Germany). For FoxP3/CD3 staining, fixed sections were permeabilized using True-Nuclear Transcription factor buffer (BioLegend, San Diego, Calif.). Sections were treated with SuperBlock (ScyTek Laboratories, Logan, Utah). PE-labeled antibody (145-2C11) to mouse CD3ε (Biolegend, San Diego, Calif.) and AF488-labeled antibody (MF-14) to mouse FoxP3 (BioLegend) were used to perform double staining, followed by 4′,6-diamidino-2-phenylindole (DAPI) (BD Biosciences) nuclear staining. For other tissue stainings, mouse and human skin sections were fixed in cold acetone. Mouse skin sections were blocked with SuperBlock and then incubated with either antibody H-90 to TRP-1 (Santa Cruz Biotechnology, Dallas, Tex.) followed by Alexa Fluor 555 labelled donkey anti-rabbit antibody (abcam), or PE-labeled MB3.6 to GD3 (Santa Cruz Biotechnology), or PE-labeled antibody YGITR 765 to GITR (Biolegend), or AF488-labeled antibody B56 to Ki67 (BD Biosciences), all followed by DAPI nuclear staining. Human skin sections were blocked with 10% normal human serum (Gemini Bio Products, West Sacramento, Calif.) and then incubated with Ta99 to TRP-1 (BioLegend) or R24 to GD3 (Abcam, Cambridge, UK). Both were detected by an HRP-conjugated goat anti-mouse IgG antibody (Agilent Dako, Santa Clara, Calif.). These stainings were developed using AEC substrate (Abcam) and nuclei were subsequently detected by incubation in Mayer's hematoxylin (Sigma-Aldrich) and blued in Scott's tap water (Sigma-Aldrich). Cells were quantified using Adobe Photoshop software. As shown in FIG. 7, TRP1+ melanocytes were maintained in the presence of Tregs. As shown in FIG. 8, CD3/FoxP3 Tregs were more abundant in the skin of mice after adoptive cell transfer.

Next, GD3 expression itself was assessed in skin biopsies from perilesional biopsies taken from actively depigmenting skin. Marked expression of GD3 was observed in human vitiligo perilesional epidermis, while melanocytes were absent from the border biopsy section (data not shown). Epidermal GD3 expression was not observed in healthy control skin, whereas melanocytes were readily detectable in this tissue (data now shown). Similarly, GD3 expression was found in depigmenting h3TA2 mouse skin (data not shown).

Summary:

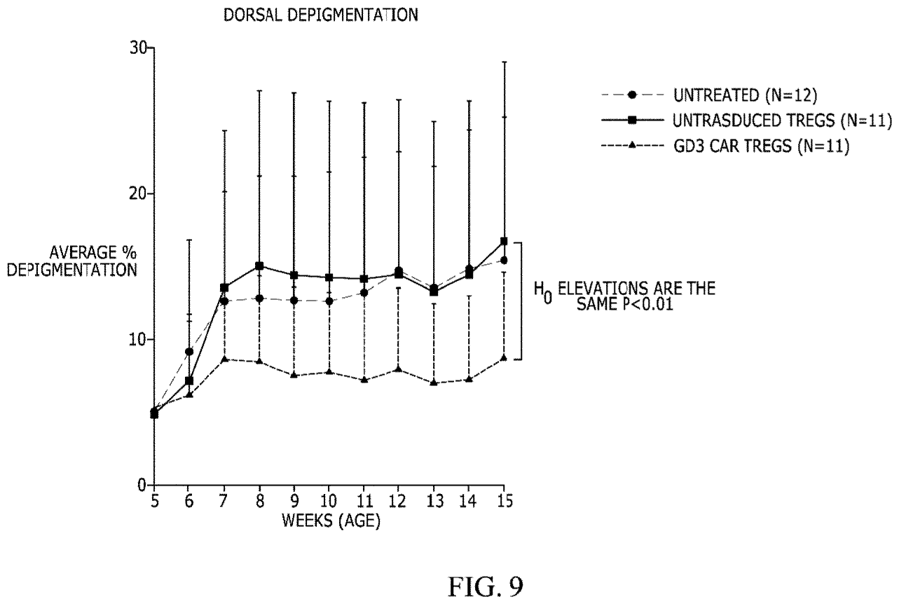

The antigen specific GD3 CAR transduced Tregs (but not untransduced Tregs) were capable of significantly suppressing depigmentation in an aggressive vitiligo mouse model of rapid, spontaneous depigmentation for the full duration of treatment. See FIG. 9. The treatment consisted of four systemic biweekly applications of 2×10⁵ transduced Tregs per 25 g mouse, and mice were followed for 10 weeks total, starting at 5 weeks of age. This is of interest to the vitiligo patient populations where effector T cells are recruited to the skin, and resident memory T cells are activated during disease activity. At that time, antigen-specific Treg can temper ongoing immunity and provide an effective intervention until T cell activity subsides.

### Example 2—High Viral Transduction of Tregs was Achieved with GD3-Encoded CAR Construct

To generate therapeutic Tregs that will engage in suppressive activity where needed, we generated FoxP3⁺CD4⁺ T cells and transduced them to express a GD3-reactive CAR as described in Example 1. In a representative example, approximately 1.5×10⁶ naïve CD4⁺ T cells were isolated from 3×10⁸ splenocytes, maintained in presence of TGF-β, and successfully polarized and amplified to approximately 1.6×10⁷ Tregs per donor mouse. TGF-β-polarized naïve CD4⁺ T cells were retrovirally transduced and GD3 CAR expression was evaluated by flow cytometry. Results showed that 86.6% of total CD4⁺ T cells were successfully transduced with the GD3 CAR construct (data not shown). After further expansion, 64±3.5% transduced cells were FoxP3⁺ Tregs. From an initial pre-expansion and transduction pool of 4×10⁶FoxP3⁺ Tregs, 2.1×10⁷ GD3 CAR-expressing, FoxP3⁺ Tregs were generated. It is contemplated that the majority of the resulting CAR transduced Tregs will function as immunosuppressive T cells, and exert a local, immunosuppressive function. Next, GD3 CAR Treg function in vitro.

### Example 3—Antigen-Specificity Increases Immunosuppressive Cytokine Production

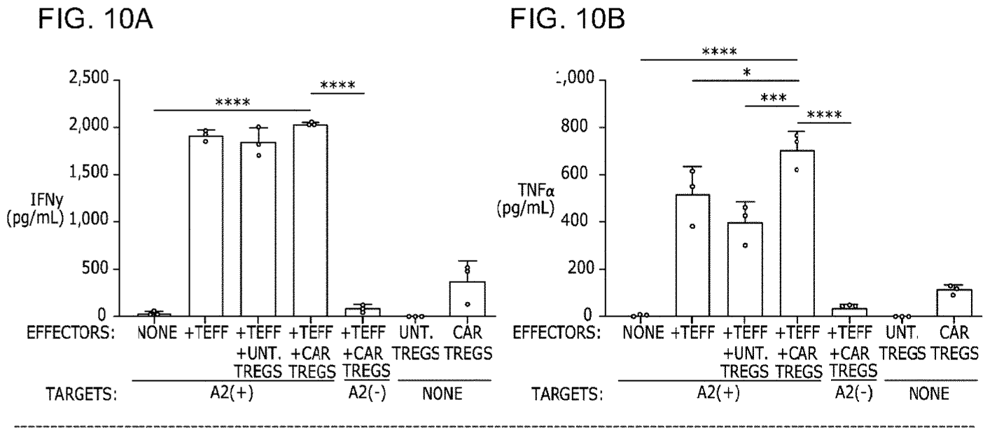

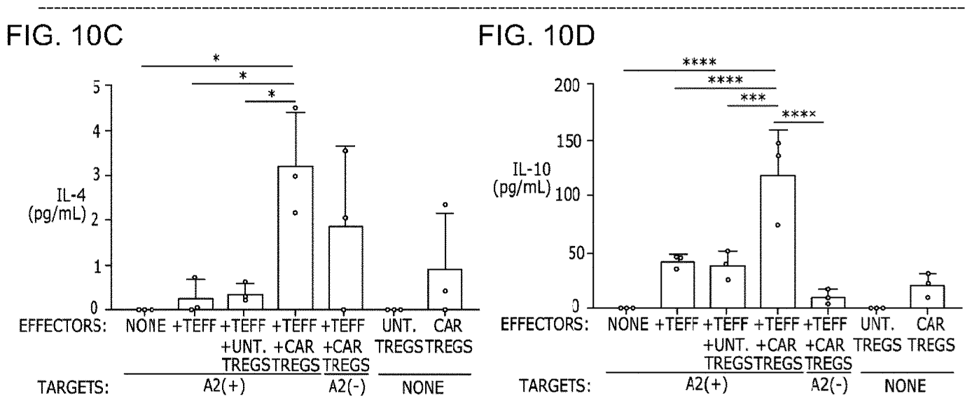

Production of representative cytokines IFN-γ, TNF-α, IL-4 and IL-10, relevant to immune activation or immunosuppression, was measured in co-cultures of GD3 CAR Tregs or untransduced Tregs with tyrosinase-reactive h3T effector T cells (Teffs) and their HLA-matched targets (1:10:1), measuring concentrations 42 hours after cells were combined in culture in presence of IL-2 (FIGS. 10A-10D). Human melanocytes can be recognized by these Teffs (Mehrotra et al. 2012). No significant differences in IFN-γ production were found in combinations that do or do not contain Tregs, suggesting that the latter had little influence on the production of this cytokine at this Treg to Teff ratio (FIG. 10A). Significantly more TNF-α (FIG. 10B, p=0.0005), IL-4 (FIG. 10C, p=0.03), and IL-10 (FIG. 10D, p=0.0005) was produced in combinations with CAR Tregs, though overall IL-4 production remained consistently low. Importantly, increased IL-10 regulatory cytokine production was observed only in presence of cytotoxic T cells and HLA-matched human melanocytes. Taken together, the cytokine environment suggests a greater immunosuppressive ability in presence of antigen-specific Tregs, stimulated by activated effector T cells. To determine whether the cytokine environment would translate to greater protection of melanocyte target cells from cell death in vitro, sustained target cell viability was measured in these co-cultures of melanocytes, Teff and Tregs.

### Example 4—Antigen-Specificity Increases the Immunosuppressive Activity of Tregs In Vitro

Tregs suppress conventional T cells via cytokines, by cell-to-cell contact or through bystander effects (Schmidt, Oberle, and Krammer 2012). To measure the resulting suppressive activity, sustained melanocyte viability was evaluated in co-cultures of targets, Teffs, and Tregs in vitro for 36 hrs. As shown in FIG. 11, the viability of targeted HLA-A2⁺ human melanocytes in different combinations of targets, Teffs and Tregs 1:10:1. The number of viable targets increased slightly over time in absence of Teff cells. In comparison, 82.2% cytotoxicity (p<0.0001) was observed in presence of effector T cells after 36 hours. Untransduced Tregs offered 35.8% (p=0.02) protection from cytotoxicity over time. A two-way ANOVA was performed with aligned rank transformation using R-software, and pairwise post-hoc multiple comparison testing according to Tukey to determine that in presence of CAR Tregs, cytotoxicity towards melanocytes was 62.0% prevented (p=0.0004). Images representing each combination of cells including targets alone, targets and Teff, and the latter combination in presence of polyclonal Tregs or CAR Tregs at different time points (data not shown) likewise reveal most inhibition of cytotoxicity in a combination that includes GD3 CAR Tregs. Thus, both untransduced Tregs and GD3 CAR Tregs offered significant protection of melanocyte viability. Importantly, the protection offered by GD3 CAR Tregs was significantly greater compared to untransduced Tregs (p=0.04), demonstrating the added benefit of antigen specificity to enhance immunosuppression.

### Example 5—Antigen-Specific Tregs Enhance Immunosuppression in h3T-A2 Mice

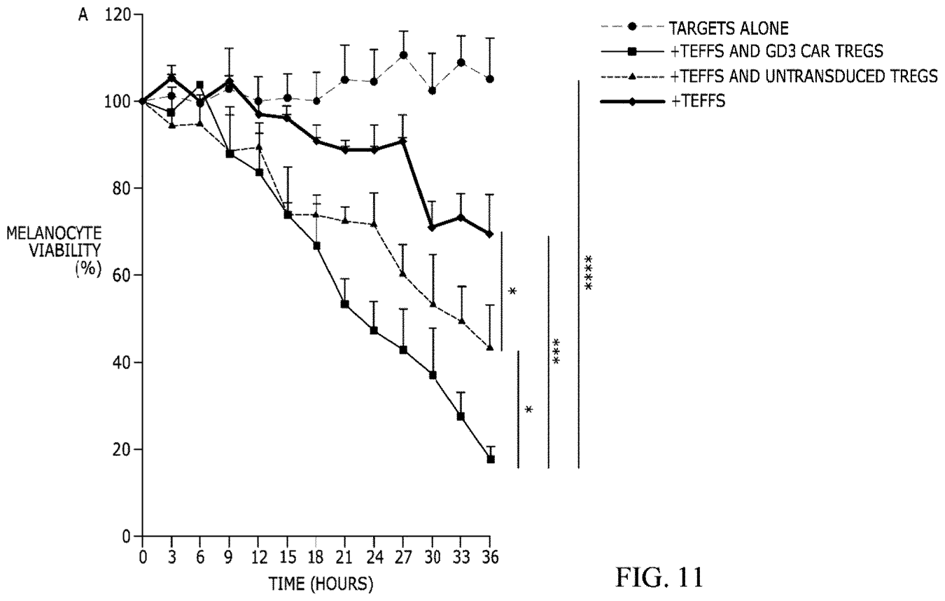

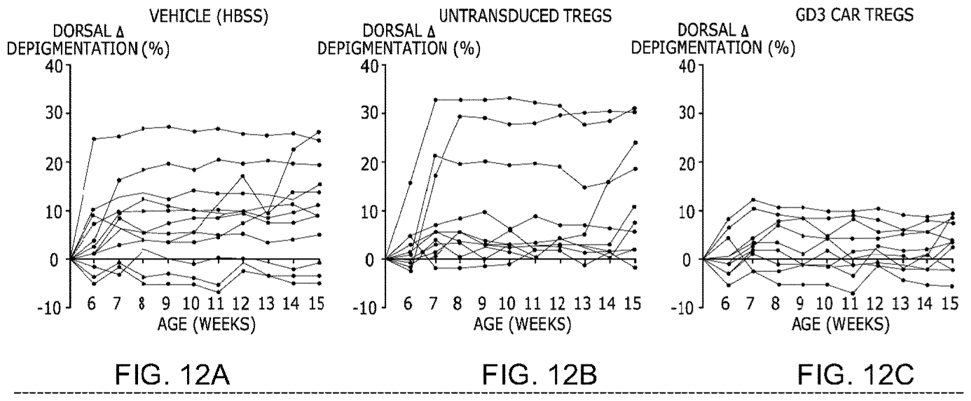

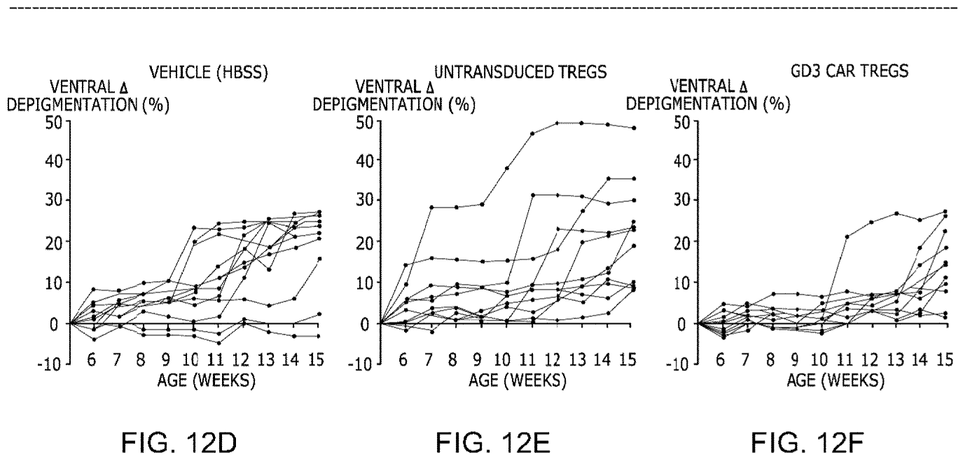

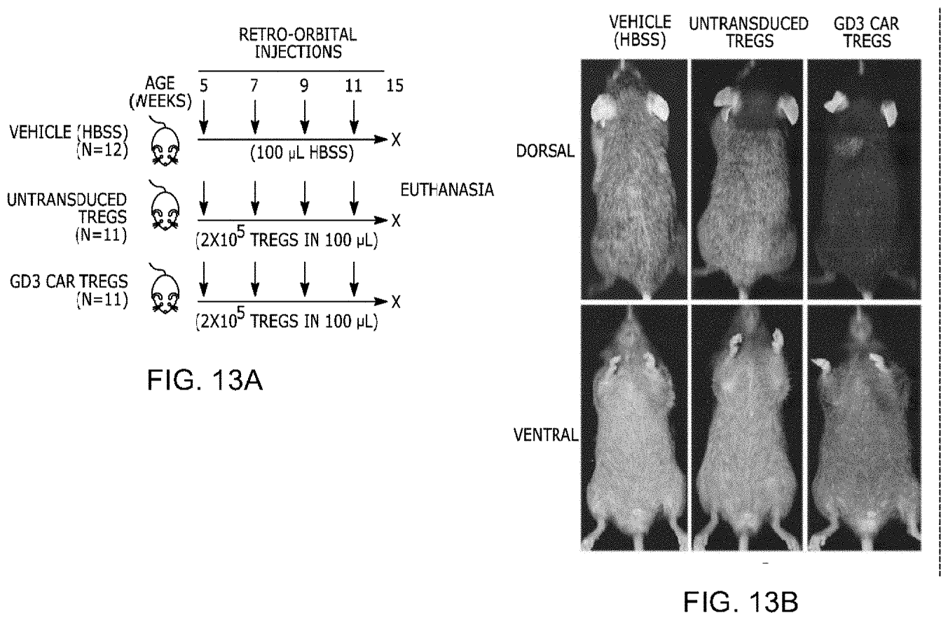

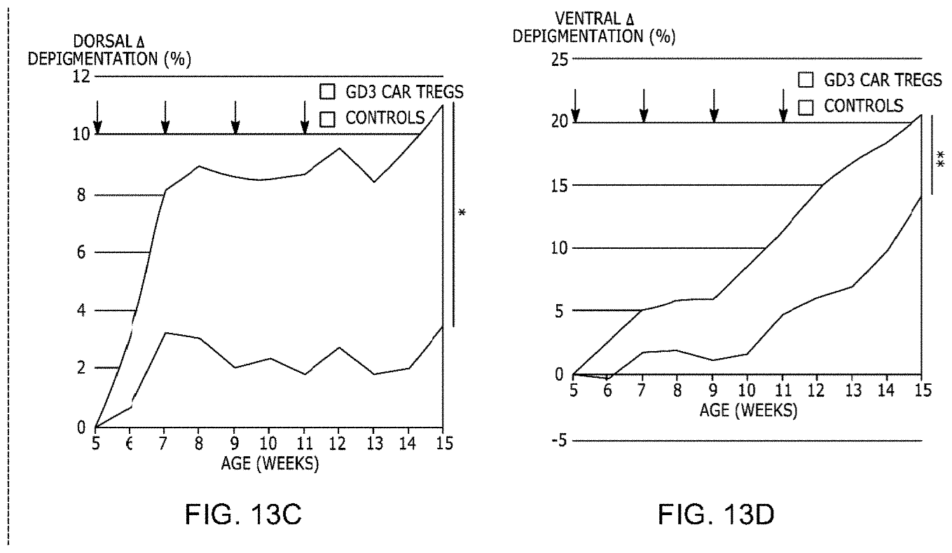

To evaluate the suppressive activity of CAR Treg in a model of progressive depigmentation, depigmentation was measured in spontaneously depigmenting h3TA2 mice starting from 5 weeks of age. Depigmentation starts shortly after birth and the animals display half-maximum depigmentation within 23 weeks (Eby et al. 2014). Mice received adoptively transferred untransduced Tregs, transduced GD3 CAR Tregs or vehicle once every two weeks for 11 weeks as outlined in FIG. 12A. Representative dorsal and ventral images of animals transfused with untransduced Tregs, GD3 CAR Tregs, or vehicle are shown in FIG. 12B. The Wilcoxon rank sum (WRS) test was used to compare the time-adjusted area under the curve (AUC) among groups. Outcomes for both vehicle and untransduced Treg control groups did not differ (dorsal p=0.97, ventral p=0.88). Therefore, the vehicle and untransduced Treg groups were merged, and compared to the GD3 CAR Treg-treated group. In a one-sided t-approximation for the WRS test, the AUC for dorsal depigmentation dropped by 73.0% (p=0.028) for CAR Treg treated mice (n=11) for the 15-week observation period. Ventral depigmentation occurs more rapidly and was evaluated separately. Here, depigmentation was significantly delayed among the CAR Treg treated group (n=11) over the follow-up period (FIG. 13C) resulting in a 60.5% reduction in the AUC (p=0.006) among CAR Treg treated mice (FIG. 13D). Individual dorsal and ventral depigmentation values for each mouse are shown in FIGS. 12A-12F. The enhanced disease control by CAR Tregs might be due to local activation of suppressive activity by GD3 expression and the presence of activated Teff on site. To assess this, changes in serum cytokine content for IFN-γ, TNF-α, IL-4 and IL-10 were measured in serum samples from mice treated with vehicle alone (n=11), untransduced Tregs (n=10), or GD3 CAR Tregs (n=9). Resulting cytokine levels were remarkably consistent among the groups at end point (data not shown). The results support the concept that Tregs, including CAR Tregs, may be preferentially activated on site in areas of immune activity. In addition, no adverse events were observed throughout the experiment, and no abnormalities were found during internal organ examination at euthanasia for mice from any groups.

### Example 6—Melanocytes are Protected in the Presence of GD3 Reactive CAR Tregs

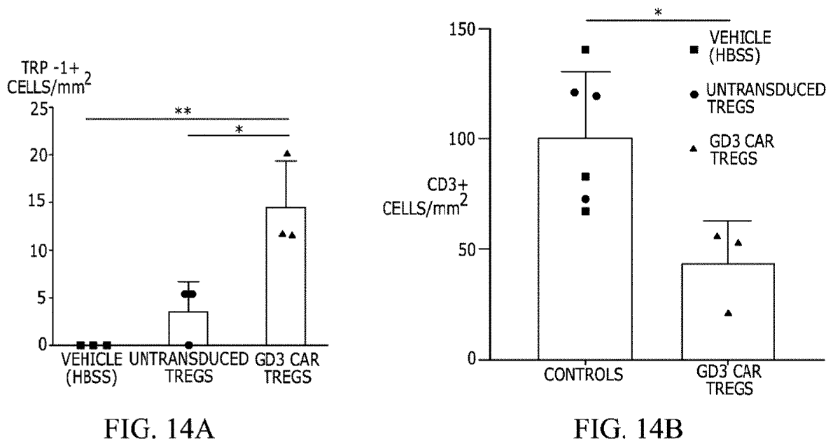

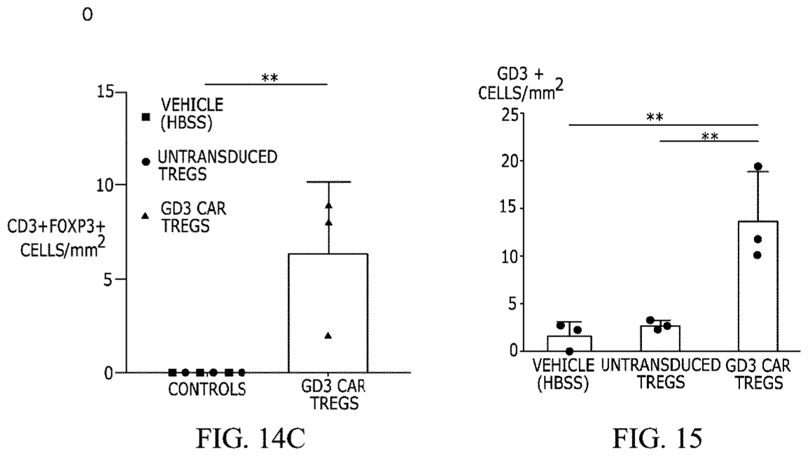

Next, mouse dorsal skin biopsies were evaluated for melanocyte abundance using antibodies to TRP-1, as shown in FIG. 14. Melanocytes were quantified as shown in FIG. 14A, where skin samples from vehicle treated mice (n=3 per group) showed complete loss of melanocytes. Skin from untransduced Treg treated mice (n=3 per group) displayed only a few remaining melanocytes, and a one-way ANOVA was performed followed by Tukey's post-hoc test to demonstrate that whereas skin from CAR Treg treated mice contained a significantly greater number of melanocytes compared to mice treated with untransduced Tregs (p=0.025), and to vehicle treated controls (p=0.006) (FIG. 15). Similar results were found when examining GD3 expression. Quantification of GD3 expressing cells revealed that mice transfused with CAR-Tregs maintained significantly more GD3 expressing cells than the vehicle HBSS-treated mice (p=0.003) or mice transfused with untransduced Treg (p=0.003). This observation supports the concept that GD3 expressing cells did not experience the cytotoxicity observed in vehicle-treated or polyclonal Treg treated mice. This confirmatory melanocyte quantification mainly corresponds with in vivo data shown in FIG. 14, demonstrating the improved suppressive ability of CAR Tregs.

### Example 7—CAR Tregs Gravitate Towards GD3 Expressing Cells in the Skin

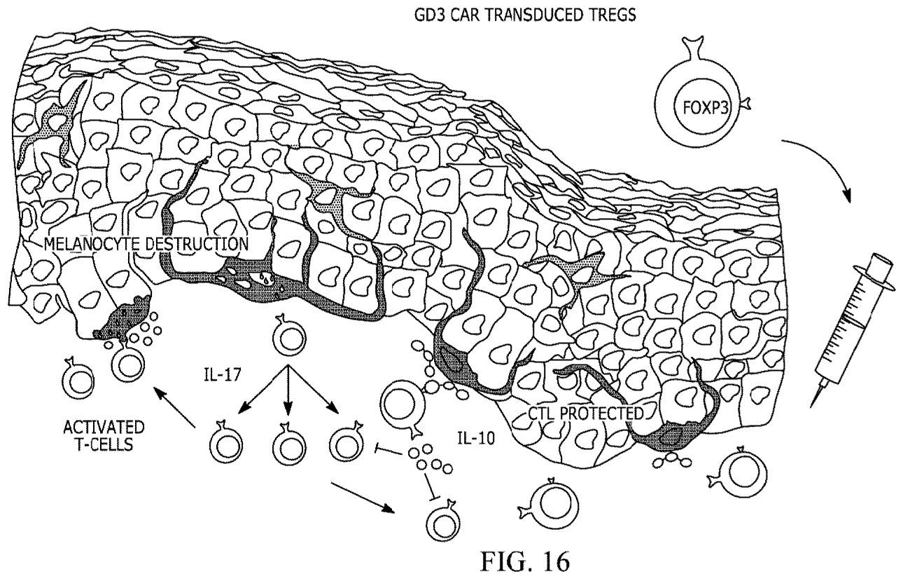

To understand whether Treg activity is correlated to the abundance of immunosuppressive T cells on site, mouse skin tissues were evaluated for T cell infiltration using antibodies to CD3ε and FoxP3. Examples of skin from the vehicle control group, and samples from the mice treated with untransduced or CAR Treg-treated mice are also shown in FIG. 14. Tregs were identified as CD3ε+ FoxP3+ cells for the same groups, respectively, overlaid with DAPI nuclear staining (data now shown). CDR3ε+ cell and CD3ε+/FoxP3+ Treg abundance was quantified as the mean±SD (at n=3 per group) for each treatment group. In a one-way ANOVA followed by Tukey's post-hoc test, the average number of infiltrating CD3ε+ T cells at end point was 2.3-fold greater (p=0.02) in the control groups as compared to the CAR Treg treated group (FIG. 14B). No (remaining) CD3+FoxP3+ Tregs were detected in either control group, whereas some CD3ε+ FoxP3+ Tregs were still detectable in skin tissue from CAR Treg treated mice 10 weeks after adoptive transfer (FIG. 14C). Evaluating Treg numbers by GITR-expression, an increase in Treg numbers at end point was again observed in skin from CAR Treg treated mice compared to those treated with untransduced Tregs (p=0.0059) or vehicle alone (p=0.0089), yet there was no difference in abundance of proliferating GITR+Ki67+ cells among groups. This data demonstrates that differences in Treg abundance may instead be defined by increased influx or decreased efflux of Tregs from the skin in CAR Treg treated mice. Nevertheless, the increased abundance of Tregs in CAR Treg treated mice at end point may explain the improved suppressive activity by CAR Tregs and suggests that maintenance of a Treg presence on site is supported by local antigen recognition (FIG. 16). In summary, the data show that antigen specificity prolonged the suppressive activity of adoptively transferred Tregs.

### REFERENCES

Ahmed et al., 2012. Pigment Cell Melanoma Res 25 (1):99-109. doi: 10.1111/j.1755-148X.2011.00920.x.
Bhardwaj et al., 2020. Int J Dermatol 59 (4):474-481. doi: 10.1111/ijd.14766.
Birklé, et al., 2000. J Neurochem 74 (2):547-54. doi: 10.1046/j.1471-4159.2000.740547.x.
Bluestone, et al., 2015.Sci. Transl Med 7 (315):315ra189. doi: 10.1126/scitranslmed.aad4134.
Bluestone, J. A., and Q. Tang. 2018. “T(reg) cells—the next frontier of cell therapy.” Science 362 (6411):154-155. doi: 10.1126/science.aau2688.
Boroughs, et al., 2019. JCI Insight 4 (8). doi: 10.1172/jci.insight.126194.
Brusko, et al., 2010. PLoS One 5 (7):e11726. doi: 10.1371/journal.pone.0011726.
Chatterjee, et al., 2014. J Invest Dermatol 134 (5):1285-1294. doi: 10.1038/jid.2013.540.
Chmielewski, et al., 2017. Transl Res 187:53-58. doi: 10.1016/j.trs1.2017.06.009.
Denman, et al., 2008. Journal of Investigative Dermatology 128 (8):2041-2048. doi: https://doi.org/10.1038/jid.2008.45.
Dwivedi, et al., 2013. Pigment Cell Melanoma Res 26 (4):586-91. doi: 10.1111/pcmr.12105.
Eby, et al., 2014. Pigment Cell Melanoma Res 27 (6):1075-85. doi: 10.1111/pcmr.12284.
Eby, et al., 2015. Invest Dermatol 135 (6):1574-1580. doi: 10.1038/jid.2015.26.
Ezzedine, et al., 2015. Lancet 386 (9988):74-84. doi: 10.1016/s0140-6736(14)60763-7.
Ferreira, et al., 2019. “Nat Rev Drug Discov 18 (10):749-769. doi: 10.1038/s41573-019-0041-4.
Frisoli, et al. 2020. Annu Rev Immunol. doi: 10.1146/annurev-immunol-100919-023531.
Golshayan, et al., 2007. Blood 109 (2):827-35. doi: 10.1182/blood-2006-05-025460.
Grimes, et al., 2018. Int J Womens Dermatol 4 (1):32-37. doi: 10.1016/j.ijwd.2017.11.005.
Guo, et al., 2015. Cell Immunol 295 (1):19-28. doi: 10.1016/j.cellimm.2015.02.006.
Henning, et al., 2018. J Invest Dermatol 138 (12):2531-2539. doi: 10.1016/j.jid.2018.06.186.
Henning, et al., 2020. PLoS One 15 (1):e0227909. doi: 10.1371/journal.pone.0227909.
Hippen, et al., 2011. Sci Transl Med 3 (83):83ra41. doi: 10.1126/scitranslmed.3001809.
Hua, et al., 2018. Sci Rep 8 (1):7059. doi: 10.1038/s41598-018-25384-x.
Huang, et al, 2001. J Invest Dermatol 116 (2):305-12. doi: 10.1046/j.1523-1747.2001.01242.x.
Jacquemin, et al., 2017. Br J Dermatol 177 (5):1367-1375. doi: 10.1111/bjd.15550.
Jin, et al., 2012. J Invest Dermatol 132 (6):1730-3. doi: 10.1038/jid.2012.37.
Jones, et al., 2014. Pharmacol 5:254. doi: 10.3389/fphar.2014.00254.
Junghans, Richard P. 2017. Cancer Gene Therapy 24 (3):89-99. doi: 10.1038/cgt.2016.82.
Karagaiah, et al., 2020. Expert Opin Emerg Drugs 25 (1):7-24. doi: 10.1080/14728214.2020.1712358.
Kemminer, et al., 2001. Biotechnol Prog 17 (5):809-21. doi: 10.1021/bp010068y.
Klarquist, et al., 2010. Pigment Cell Melanoma Res 23 (2):276-86. doi: 10.1111/j.1755-148X.2010.00688.x.
Kniep, et al., 1993. Blood 82 (6):1776-86.
Koristka, et al., 2018. J Autoimmun 90:116-131. doi: 10.1016/j.jaut.2018.02.006.
Kundu, et al., 2019. Exp Dermatol 28 (6):647-655. doi: 10.1111/exd.13677.
Le Poole, I. C. and R. M. Luiten. 2008. Curr Dir Autoimmun 10:227-43. doi: 10.1159/000131485.
Le Poole, I. C. and S. Mehrotra. 2017. J Investig Dermatol Symp Proc 18 (2):538-s45. doi: 10.1016/j.jisp.2016.10.023.
Le Poole, I. C et al., 2003. Br J Dermatol 149 (4):739-48. doi: 10.1046/j.1365-2133.2003.05539.x.
Le Poole, I. C., et al., 2004. J Investig Dermatol Symp Proc 9 (1):68-72. doi: 10.1111/j.1087-0024.2004.00825.x.
Levings, et al., 2001. J Exp Med 193 (11):1295-302. doi: 10.1084/jem.193.11.1295.
Lili, et al., PLoS One 7 (5):e37513. doi: 10.1371/journal.pone.0037513.
Liu, et al., 2017. J Am Acad Dermatol 77 (4):675-682.el. doi: 10.1016/j.jaad.2017.05.043.
Lo, et al., 2010a. Clin Cancer Res 16 (10):2769-80. doi: 10.1158/1078-0432.Ccr-10-0043.
Lo, et al., 2010b. Clinical Cancer Research 16 (10):2769. doi: 10.1158/1078-0432.CCR-10-0043.
MacDonald, et al., 2019. Cytotherapy 21 (12):1216-1233. doi: 10.1016/j.jcyt.2019.10.011.
Marek-Trzonkowska, et al., 2014. Clin Immunol 153 (1):23-30. doi: 10.1016/j.clim.2014.03.016.
Marek-Trzonkowska, et al., 2012. Diabetes Care 35 (9):1817-20. doi: 10.2337/dc12-0038.
Marek-Trzonkowska, et al, 2013. Pediatr Diabetes 14 (5):322-32. doi: 10.1111/pedi.12029.
Mathew, et al., 2018. Sci Rep 8 (1):1136. doi: 10.1038/s41598-018-19621-6.
Mehrotra, et al., 2012. J Immunol 189 (4):1627-38. doi: 10.4049/jimmunol.1103271.
Mosenson, et al., 2014. Pigment Cell Melanoma Res 27 (2):209-20. doi: 10.1111/pcmr.12208.
Nakano, et al., 1999. J Investig Dermatol Symp Proc 4 (2):173-6. doi: 10.1038/sj.jidsp.5640204.
Noyan, et al., 2013. Transplant Proc 45 (5):1832-7. doi: 10.1016/j.transproceed.2013.01.073.
Oren, et al., 2014. J Immunol 193 (11):5733-43. doi: 10.4049/jimmunol.1301769.
Osinubi, et al., 2018. Br J Dermatol 178 (4):863-878. doi: 10.1111/bjd.16049.
Paller, et al., 1993. J Invest Dermatol 100 (6):841-5. doi: 10.1111/1523-1747.ep12476755.
Putnam, et al., 2009. Diabetes 58 (3):652-62. doi: 10.2337/db08-1168.
Raffin, et al., 2020. Nat Rev Immunol 20 (3):158-172. doi: 10.1038/s41577-019-0232-6.
Raffin, et al., 2016. The Journal of Immunology 196 (1 Supplement):210.19.
Rossignoli, et al., 2019. Cancer Gene Ther 26 (1-2):11-16. doi: 10.1038/s41417-018-0034-1.
Safinia, et al., 2016. Oncotarget 7 (7):7563-77. doi: 10.18632/oncotarget.6927.
Sagoo, et al., 2011. Sci Transl Med 3 (83):83ra42. doi: 10.1126/scitranslmed.3002076.
Schmidt, A., N. Oberle, and P. H. Krammer. 2012. Front Immunol 3:51. doi: 10.3389/fimmu.2012.00051.
Serra, P., and P. Santamaria. 2015. Clin Immunol 160 (1):3-13. doi: 10.1016/j.clim.2015.02.003.
Trenado, et al., 2003. J Clin Invest 112 (11):1688-96. doi: 10.1172/jci17702.
Tsang, et al., 1995. Cytokine 7 (5):389-97. doi: 10.1006/cyto.1995.0054.
Tulic, et al., 2019. Nat Commun 10 (1):2178. doi: 10.1038/s41467-019-09963-8.
Walker, et al., 2017. Mol Ther 25 (9):2189-2201. doi: 10.1016/j.ymthe.2017.06.008.
Watanabe, et al., 2015. J Immunol 194 (3):911-20. doi: 10.4049/jimmuno1.1402346.
Weiner, et al., 2015. Transplant Direct 1 (1):1-7. doi: 10.1097/txd.0000000000000511.
Wu, et al., 2013. Cancer Res 19 (17):4728-39. doi: 10.1158/1078-0432.ccr-13-0088.
Yun, et al., 2000. Neoplasia 2 (5):449-59. doi: 10.1038/sj.neo.7900108.
Zhang, et al., 2020. J Autoimmun:102461. doi: 10.1016/j.jaut.2020.102461.

What is claimed is:

1. A method of treating of treating vitiligo in a subject in need thereof, comprising administering to the subject a regulatory T cell engineered to express a chimeric antigen receptor (CAR) that specifically binds ganglioside D3.

2. The method of claim 1, wherein administration of the cells is not toxic to TRP1+ melanocytes in the subject.

3. The method of claim 1 or claim 2, wherein the cells are autologous.

4. The method of any one of claims 1-3, wherein the subject is human.

5. The method of any one of claims 1-4, wherein administration of the cells reduces depigmentation in the skin of the subject.

6. The method of any one of claims 1-4, wherein administration of the cells results in an 50% decrease in depigmentation over the treatment period compared to subjects not receiving the cells.

7. The method of any one of claims 1-6, wherein the cells are administered by subcutaneous injection.

8. The method of any one of claims 1-6, wherein the cells are administered intravenously.

9. The method of any one of claims 1-8, wherein the subject is also suffering from alopecia, hypothyroid disease or other vitiligo-related autoimmune disease.

10. The method of any one of claims 1-9, wherein administration of the cells results in at least a 2.5 fold increase in IL-10 secretion by CAR Treg in response to relevant target cells as measured by ELISA.


SEQUENCE LISTING (2 sequences)

SEQ ID NO: 1 - protein, 140 aa, Mus sp.
MNFGFSLIFLVLVLKGVQCEVVVVESGGGFVKPGGSLKLSCAAAGFTFSRYAMSWVRQTPEKRLEWVATISSGGSHTYYPDSVKGRFTISRDNAKNTLYLQMSSLRSEDTAIYYCARPGYDRGAWFFDVWGAGTTVTVSS

SEQ ID NO: 2 - protein, 128 aa, Mus sp.
MVFTPQILGLMLFWISASRGDIVLTQSPATLSVTPGDSVSLSCRASQIISNNLHWYQQKSHESPRLLIKYASQSISGIPSRFSGSGSGTDFTLSINSVETEDFGMYFCQQSNSWPLTFGSGTKLEIKR